(19) United States
(12) Patent Application Publication
(10) Pub. No.: US 2009/0318669 A1
(43) Pub. Date: Dec. 24, 2009

(54) Novel Isodipeptide

(76) Inventor: Yoshiaki Kiso, Osaka-fu (JP)

Correspondence Address: WENDEROTH, LIND & PONACK, L.L.P., 1030 15th Street, N.W.,, Suite 400 East, Washington, DC, 20005-1503, US

(21) Appl. No.: 12/224,170

(22) PCT Filed: Feb. 16, 2007
(86) PCT No.: PCT/JP2007/052838

§ 371 Date: Aug. 20, 2008

(30) Foreign Application Priority Data
Feb. 22, 2006 (JP) 2006-044853

(51) Int. Cl. C07K 1/00

(52) U.S. Cl. 530/333

(57) ABSTRACT

An isodipeptide of the following formula (1) which is a useful synthetic unit for the effective synthetic method for a polypeptide and the like:

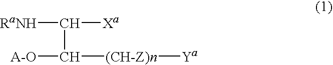

wherein A is N-protected amino acid residue, R^{a} is amino protective group, X^{a} is carboxyl group, hydrogen atom, alkyl group, aralkyl group, aryl group or heteroaryl group, Y^{a} is carboxyl group, hydrogen atom or alkyl group, Z is hydrogen atom or alkyl group, and n is an integer of 0-3, provided that either one of X^{a} and Y^{a} is carboxyl group.

### TECHNICAL FIELD

The present invention relates to a novel isodipeptide useful as a synthetic unit for a peptide and the like.

### BACKGROUND ART

The synthesis of “difficult sequence”-containing peptides is one of the most problematic areas in peptide chemistry. Such peptides are often obtained with significant low yield and purity in conventional solid-phase peptide synthesis. Furthermore, such peptides are generally rich in hydrophobic property and therefore, easily aggregate in various solvents during synthesis and purification. The reason is considered to be due to small aggregates based on β-sheet structure constituted by hydrophobic interaction and hydrogen bond between peptides.

Recently the present inventor et al., have developed “O-acyl isopeptide method” in regard to the effective synthesis of difficult sequence-containing peptides (See non-patent literatures 1 and 2.). This method relates to prepare O-acyl isopeptide by isomerizing amide bond into ester bond, and then to prepare the objective peptide by intramolecular O—N acyl migration, in case of the amino acid containing hydroxyl group such as serine residue.

However, in order to prepare the objective peptide and the like (hereinafter described as a peptide including a long or short chained peptide and a protein) in high yield and high purity by practicing this method, the higher technique is still required and further it is found that on the way of reaction, namely in esterification, for example racemization in esterified Val residue occurs in high frequency and therefore, the decrease of the yield and troublesome on purification are accompanied with.

[Non-patent literatures 1] Tetrahedron Letters 45 (2004) 5965-5968
[Non-patent literatures 2] Chem. Commun., 2004, 124-125

### DISCLOSURE OF INVENTION

The present inventor has earnestly investigated to solve the above problems and has found that by using an isodipeptide (1) described below, especially as a synthetic unit in solid-phase peptide synthesis, the objective peptide can be easily prepared in high yield and high purity. Thus the present invention was completed.

The present invention relates to an isodipeptide of the following formula (1):

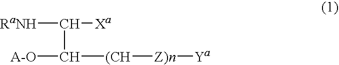

wherein A is N-protected amino acid residue, R^{a} is amino protective group, X^{a} is carboxyl group, hydrogen atom, alkyl group, aralkyl group, aryl group or heteroaryl group, Y^{a} is carboxyl group, hydrogen atom or alkyl group, Z is hydrogen atom or alkyl group, and n is an integer of 0-3, provided that either one of X^{a} and Y^{a} is carboxyl group.

### Effect of Invention

In the solid-phase peptide synthesis by using isodipeptide synthetic unit of the present invention, the desired peptide can be fully automatically synthesized and therefore, its synthesis is very easily and the present invention is very valuable from a viewpoint of the industrial application.

Furthermore, by using the isodipeptide (1) of the present invention as a synthetic unit for a peptide, it is unexpectedly found to avoid the above mentioned side reaction such as racemization and to obtain the objective peptide in high yield and purity.

### Mode for Carrying Out the Invention

The present invention relates to the isodipeptide represented by the following formula (1):

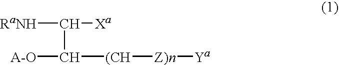

wherein A, R^{a}, X^{a}, Y^{a} and n are the same as defined above.

Alkyl group in X^{a} and Y^{a} of the formula (1) is not limited, but is preferably C₁₋₆ straight or branched chain alkyl group. Aryl or heteroaryl moiety of aralkyl group, aryl group or heteroaryl group in X^{a} of the formula (1) may be substituted by a substituent such as methyl, nitro, chlorine atom, etc.

As a preferable isodipeptide in the formula (1) wherein n is 0 or 1, an isodipeptide of the formula (1) wherein X^{a} is carboxyl group, Y^{a} is hydrogen atom or alkyl group, and n is 0, or its optical isomer, and an isodipeptide wherein X^{a} is hydrogen atom, alkyl group, aralkyl group, aryl group or heteroaryl group, Y^{a} is carboxyl group and n is 0, or its optical isomer are illustrated.

As N-protective group of an amino acid or a protective group represented by R^{a} in the formula (1) are illustrated usual protective groups of an amino acid. As preferable protective groups, there are illustrated such protective groups that ester bond in the isodipeptide (1) is not cleaved and only the object protective group is cleaved. For example, urethane type-protective groups, such as 9H-fluoren-9-ylmethoxycarbonyl (Fmoc), tert-butoxycarbonyl (Boc), benzyloxycarbonyl (Z), 2-chlorobenzyloxycarbonyl, etc., are illustrated.

In the acid residue of N-protected amino acid represented by A, when said amino acid has a functional group such as hydroxy group on its side chain and further carboxyl group, etc., such groups are preferably protected by a suitable known protective group.

The especially preferable compound in the isodipeptide (1) is an isodipeptide of the following formula (1a):

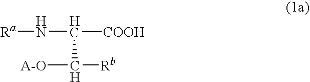

wherein A and R^{a} are the same as defined above, and R^{b} is hydrogen atom or methyl group.

This compound (1a) is especially valuable in the method for synthesizing polypeptides containing L-threonine or L-serine as a component.

The preferable compound is also an isodipeptide of the following compound (1b):

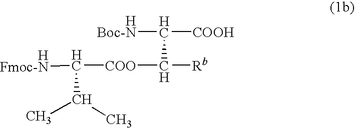

wherein Fmoc is 9H-fluoren-9-ylmethoxycarbonyl, Boc is tert-butoxycarbonyl, and R^{b} is the same as defined above.

This compound (1b) is especially valuable in the method for synthesizing a peptide containing L-valine-L-threonine or L-valine-L-serine as a dipeptide-component.

The most preferable compound in the isodipeptide (1) is the compound of the following formula (1c) (hereinafter sometimes abbreviated as “Boc-Thr(Fmoc-Val)-OH”)

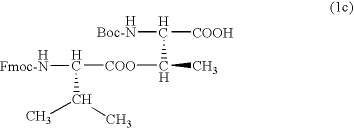

wherein Fmoc and Boc are the same as defined above.

Furthermore, the compounds derived from the isodipeptide (1) of the present invention or obtained by modification of the isodipeptide (1) and having the same function as the compound of the present invention are included in the scope of the present invention as a matter of cause.

The isodipeptide (1) of the present invention can be prepared by known esterification using a carboxylic acid and an alcohol.

Namely an isopeptide (2b) is obtained by reacting a N-protected amino acid represented by A-OH and compound (2a) prepared by protecting carboxyl group of an amino acid having a hydroxyl group on its side chain represented by the following formula (2):

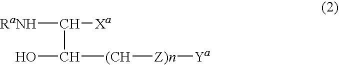

wherein R^{a}, X^{a}, Y^{a}, z and n are the same as defined above, and then, by deprotecting only the protective group of the carboxyl group of the said compound (2b) to give the isodipeptide (1).

The compounds of the formula (2) preferably include for example, an amino acid having hydroxyl group on its side chain, such as threonine, serine, statine, norstatine, etc.,

N-protected amino acid represented by A-OH is not limited and includes various typed amino acids such as α or β-amino acid having N-protected amino group.

When N-protected amino acid represented by A-OH has a functional side chain, in order to avoid side reaction, the group is preferably protected by the known protecting method.

The above esterification is carried out by the conventional method. The solvent used includes chloroform, dichloromethane, etc. The reaction temperature depends on the starting materials, but is usually around 25° C.

The protective group of carboxyl group of the compound (2a) preferably includes groups which are removed by hydrogenation such as benzyl group, p-nitrobenzyl group, or 4-pyridylmethyl group in a viewpoint of avoidance of cleavage of the ester part. Furthermore, from this viewpoint the above mentioned amino protective group of isodipeptide (1) preferably includes the protective groups which are not removed by hydrogenation.

The removal of carboxyl group of compound (2b), an intermediate is carried out, for example by introducing hydrogen gas in the presence of Pd/C.

Thus obtained isodipeptide (1) is conventionally isolated, and purified to give the purified product. The purified product is served as a synthetic unit for preparing a desired polypeptide.

A-OH and compound (2) which are starting materials may have one or more asymmetric carbons and therefore, the objective isodipeptide can be obtained in the form of optical isomer, racemic compound thereof, diastereomer or the mixture thereof, depending on the kind of starting materials. For example, by using a respective optical isomer, there is obtainable an isodipeptide in diastereomer type (See (1b) and (1c).).

When the product is obtained in racemic mixture or diastreomer mixture, the product is conventionally optically resoluted and purified along the lines of the object to give a desired isodipeptide with highly optical purity.

Taking a pentapeptide, Ac-Val-Val-Thr-Val-Val-NH₂ (3a) as an example, and using Boc-Thr(Fmoc-Val)-OH (1c) as a synthetic unit, the synthetic scheme in accordance with the solid-phase peptide synthesis is shown below.

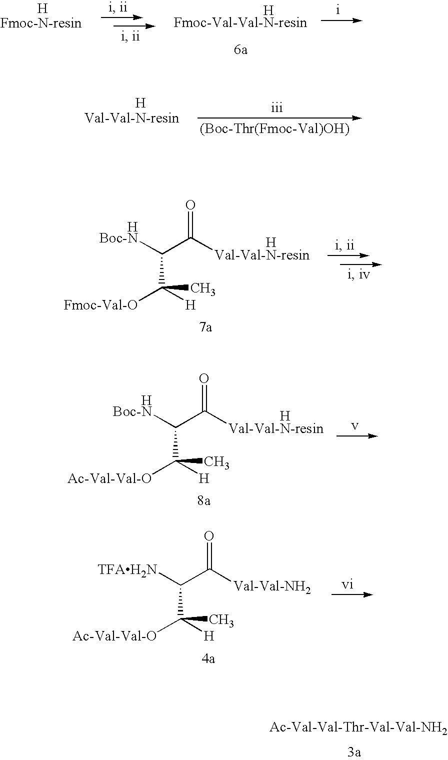

(In the above formulas, Fmoc and Boc are the same as defined above, TFA is tetrafluoroacetic acid and all of the amino acids are L-form.)

The reaction conditions in each step of the above reaction route are as follows:

Reaction i: 20% piperidine/DMF for 20 minutes.
Reaction ii: Fmoc-Val-OH (2.5 eq), 1,3-diisopropylcarbodiimide (2.5 eq),

1-hydroxybenzotriazole (2.5 eq) in DMF for 2 hrs.

Reaction iii: Boc-Thr(Fmoc-Val)OH (2.5 eq), 1,3-diisopropylcarbodiimide)

(2.5 eq), 1-hydroxybenzotriazole (2.5 eq) in DMF for 2 hrs.

Reaction iv: Ac₂O (1.5 eq), triethylamine (1.0 eq) in DMF for 2 hrs.

### Reaction v: TFA-m-cresol-thioanisole-H₂O 1.5 hrs.

Reaction vi: phosphate buffer saline solution, pH 7.4 (25° C.).

Instead of the above Fmoc-Val-OH, using other amino acid protected by Fmoc, etc., there are obtained desired various difficult sequence-containing peptides. Coupling reaction of other amino acid against an amino acid or a peptide is carried out by the conventional method in peptide synthesis. O—N intramolecular acyl migration reaction on ester (Reaction vi) is carried out by the known method (See non-patent literature 1.).

The present invention is further explained by the following examples, but should not be limited by these examples.

### EXAMPLE 1

### Synthesis of Boc-Thr(Fmoc-Val)-OH

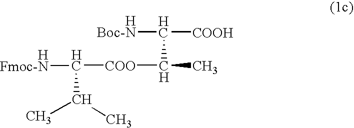

N-(t-Butoxycarbonyl)-L-threonine benzyl ester (Boc-Thr-OBzl) (139 mg, 0.449 mmol) was dissolved in dry CHCl₃ (10 mL), and thereto were added at 0° C. N-(9H-fluoren-9-ylmethoxycarbonyl)-L-valine (Fmoc-Val-OH) (183 mg, 0.539 mmol),4-dimethylaminopyridine (5.5 mg, 0.045 mmol) and 1-ethyl-3-(3′-dimethylaminopropyl)carbodiimide HCl (104 mg, 0.539 mmol), respectively. The reaction mixture was gradually warmed to room temperature in a period of 2 hours and stirred at the same temperature for 5 hours. After diluted with AcOEt, the solution was washed with H₂O, 1M HCl, saturated NaHCO₃ and saturated brine, dried over MgSO₄ and concentrated in vacuo. The resulting residue was purified by silica gel chromatography (AcOEt:hexane 1:4) to give Boc-Thr(Fmoc-Val)-OBzl (266 mg, 0.422 mmol, yield 94%). In the above reaction racemization was not observed (This was confirmed by separately synthesizing D-valine derivatives.). Then to Boc-Thr(Fmoc-Val)-OBzl (236 mg, 0.374 mmol) in AcOEt (10 mL) was added Pd/C (12 mg) and the mixture was vigorously stirred for 3 hours. After removal of Pd/C through Celite, the solvent was concentrated in vacuo, filtered by silica gel (AcOEt:hexane 1:2) and washed with MeOH to give objective Boc-Thr(Fmoc-Val)-OH (3a) with high purity (186 mg, 0.346 mmol, yield 92%).

HRMS (FAB): calcd. for C₂₉H₃₆N₂O₈Na (M+Na)⁺: 563.2369, found: 563.2373; HPLC analysis at 230 nm: purity 95% or more; NMR (CD3OD, 400 MHz): δ 7.79 (d, ³J(H,H)=7.3 Hz, 2H, CH), 7.75-7.66 (m, 2H, CH), 7.38 (t, ³J(H,H)=7.5 Hz, 2H, CH), 7.33-7.29 (m, 2H, CH), 5.44-5.41 (m, 1H, CH), 4.38 (d, ³J(H,H)=7.0 Hz, 2H, CH₂), 4.25-4.22 (m, 2H, CH), 4.05-4.01 (m, 1H, CH), 2.11-2.02 (m, 1H, CH), 1.44 (s, 9H, CH₃), 1.25 (d, ³J(H,H)=6.4 Hz, 3H, CH₃), 0.91, 0.89 (2d, ³J(H,H)=7.7, 7.0 Hz, 6H, CH₃).

### EXAMPLE 2

Instead of Boc-Thr-OBzl, using N-(t-butoxycarbonyl)-L-serine benzyl ester (Boc-Ser-OBzl) in accordance with the method of the above example 1, there can be obtained Boc-Ser(Fmoc-Val)-OH.

### EXAMPLE 3

### Synthesis of Ac-Val-Val-Thr-Val-Val-NH₂ (3a)

Using Rink Amide AM resin (100 mg, 0.071 mmol), after washing this resin with DMF (1.5 mL×5), according to the sequence by using Fmoc-Val-OH there was constructed H-Val-Val-NH-resin by the conventional method. This product was condensed with Boc-Thr(Fmoc-Val)-OH (100 mg, 0.18 mmol) which was synthesized by example 1 in DMF (1.5 mL) in the presence of 1,3-diisopropylcarbodiimide (29.0 μL, 0.18 mmol) and 1-hydroxybenzotriazole (28.4 mg, 0.18 mmol). Then after introducing Fmoc-Val-OH (62.8 mg, 0.18 mmol) thereto, N-acetylation was carried out by using Ac₂O (10.5 μL, 0.11 mmol)-triethylamine (10.4 μL, 0.071 mmol). The protected peptide resin was stirred under the presence of thioanisole (66.7 μL), m-cresol (66.7 μL) and water (66.7 μL) in TFA (2.47 mL) for 90 minutes. The reaction solution was concentrated, washed with Et₂O, suspended in water and lyophilized to give O-acyl isopeptide (4a) of the following formula:

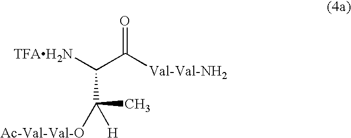

as white amorphous powders (21.2 mg, yield 44.5%).

HRMS (FAB): calcd for C₂₆H₄₉N₆O₇ (M+H)⁺: 557.3663, found: 557.3666; HPLC analysis at 230 nm: purity 95% or more.

As H-Thr-Val-Val-NH₂ was not detected in this reaction products, it is estimated that ester bond formed is stable in piperidine or by TFA treatment, and in case of de-Fmoc reaction in the last Val, diketopiperazine-formation was not proceeded.

The above obtained O-acyl isopeptide (4a) (3.0 mg) was dissolved in phosphate buffer saline solution (pH 7.4) (3 mL) and the solution was stirred at room temperature overnight. The resulting white deposite was filtered, washed with H₂O and MeOH, and dried in vacuo to give Ac-Val-Val-Thr-Val-Val-NH₂ (3a) as white powders (yield 2.4 mg (96%)).

HRMS (FAB): calcd. for C₂₆H₄₉N₆O₇ (M+H)⁺: 557.3663, found: 557.3667; HPLC analysis at 230 nm: Purity 95% or more; retention time of the obtained compound on HPLC (0-100% CH₃CN 40 minutes 230 nm) was correspond to that of peptide (3a) conventionally synthesized.

Compound (4a) was stable at 4° C. at least for 2 years. On the other hand, in case that compound (4a) is dissolved in phosphate buffer saline solution (pH 7.4) and stirred at room temperature, quantitative O—N intramolecular acyl migration to peptide (3a) was confirmed without side reaction.

### INDUSTRIAL APPLICABILITY

By using the isodipeptide (1) of the present invention as a peptide synthesis-unit, the desired peptide can be fully automatically obtainable by solid-phase peptide synthesis as well as to avoid side reaction such as racemization, etc.

1-10. (canceled)

11. In a process for preparing a peptide containing an amino acid having hydroxy group on its side chain, the process for preparing the peptide which is characterized in using an isodipeptide (1) represented by the following formula (1) as its synthetic unit, 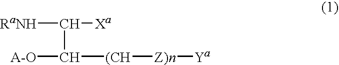
wherein A is N-protected amino acid residue, R^{a} is amino protective group, X^{a} is carboxyl group, hydrogen atom, alkyl group, aralkyl group, aryl group or heteroaryl group, Y^{a} is carboxyl group, hydrogen atom or alkyl group, Z is hydrogen atom or alkyl group, and n is an integer of 0-3, provided that either one of X^{a} and Y^{a} is carboxyl group.

12. In a process for preparing a peptide containing L-threonine or L-serine as the polypeptide component, the process for preparing the peptide which is characterized in using an isodipeptide represented by the following formula (1a) as its synthetic unit, 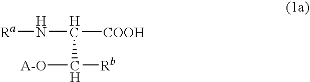
wherein A is N-protected amino acid residue, and R^{a} is amino protective group, and R^{b} is hydrogen atom or methyl group.

13. In a process for preparing a peptide containing L-valine-L-threonine or L-valine-L-serine as the peptide component, the process for preparing the peptide which is characterized in using an isodipeptide represented by the following formula (1b) as its synthetic unit, 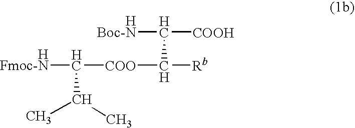
wherein Fmoc is 9H-fluoren-9-ylmethoxycarbonyl group, Boc is tert-butoxycarbonyl group and R^{b} is hydrogen atom or methyl group.

14. The process for preparing the peptide according to claim 11, which is characterized in using the isodipeptide, wherein in the formula (1) n is 0 or 1.

15. The process for preparing the peptide according to claim 11, which is characterized in using the isodipeptide, wherein in the formula (1) X^{a} is carboxyl group, Y^{a} is hydrogen atom or alkyl group, and n is 0.

16. The process for preparing the peptide according to claim 11, which is characterized in using the isodipeptide, wherein in the formula (1) X^{a} is hydrogen atom, alkyl group, aralkyl group, aryl group, or heteroaryl group, Y^{a} is carboxyl group, and n is 0.

17. In a process for preparing a peptide containing L-valine-L-threonine or L-valine-L-serine as the peptide component, the process for preparing the peptide which is characterized in using an isodipeptide represented by the following formula (1c) as its synthetic unit, 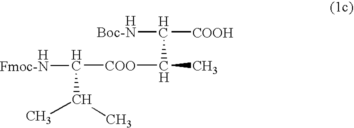
wherein Fmoc is 9H-fluoren-9-ylmethoxycarbonyl group and Boc is tert-butoxycarbonyl group.

18. In a process for preparing a peptide containing an amino acid having hydroxy group on its side chain, use of an isodipeptide (1) represented by the following formula (1) as its synthetic unit, 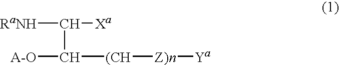
wherein A is N-protected amino acid residue, R^{a} is amino protective group, X^{a} is carboxyl group, hydrogen atom, alkyl group, aralkyl group, aryl group or heteroaryl group, Y^{a} is carboxyl group, hydrogen atom or alkyl group, Z is hydrogen atom or alkyl group, and n is an integer of 0-3, provided that either one of X^{a} and Y^{a} is carboxyl group.

19. In a process for preparing a peptide containing L-threonine or L-serine as the peptide component, use of an isodipeptide represented by the following formula (1a) as its synthetic unit, 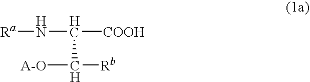
wherein A is N-protected amino acid residue, and R^{a} is amino protective group, and R^{b} is hydrogen atom or methyl group.

20. In a process for preparing a peptide containing L-valine-L-threonine or L-valine-L-serine as the peptide component, the process for preparing the peptide which is characterized in using an isodipeptide represented by the following formula (1b) as its synthetic unit, 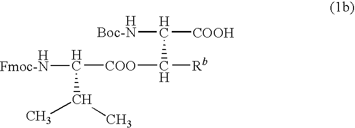
wherein Fmoc is 9H-fluoren-9-ylmethoxycarbonyl group, Boc is tert-butoxycarbonyl group and R^{b} is hydrogen atom or methyl group.


SEQUENCE LISTING (1 sequence)

SEQ ID NO: 1 - protein, 5 aa, Artificial Sequence
  Synthetic Construct
VVTVV